(19) United States
(12) Patent Application Publication
(10) Pub. No.: US 2026/0144708 A1
(43) Pub. Date: May 28, 2026

(54) VISUAL AID FOR ASSISTING A USER IN THE PREPARATION OF A PLURALITY OF SYRINGES

(71) Applicant: Genzyme Corporation, Cambridge, MA (US)

(72) Inventors: Martin Michael Coyne, III, Boonton, NJ (US); Madeleine Hayden Anderson, Boonton, NJ (US); Katherine Ford Brigham, Boonton, NJ (US); Alejandra Linares Martinez, Boonton, NJ (US); Matthew Conner D'Auria, Boonton, NJ (US)

(21) Appl. No.: 18/960,725

(22) Filed: Nov. 26, 2024

(51) Int. Cl. A61J 1/20

(52) U.S. Cl. CPC A61J 1/2003

(57) ABSTRACT

The present disclosure relates to a visual aid for assisting a user in the preparation of a plurality of syringes, the visual aid comprising a plurality of images, wherein each image: is visually representative of a respective syringe of the plurality of syringes; and comprises a respective first visual feature that is representative of a respective first visual feature of the respective syringe and a respective second visual feature that is representative of a respective second visual feature of the respective syringe, the visual features arranged such that they indicate a respective volume of a medicament to be drawn into the respective syringe. The present disclosure also relates to a system comprising a visual aid and a plurality of syringes. The present disclosure also relates to a method of using a visual aid.

### TECHNICAL FIELD

This application relates to a visual aid for assisting a user in the preparation of a plurality of syringes, for example to assist the user in drawing a predetermined volume of a medicament into the syringes.

### BACKGROUND

Titration refers to a process of gradually adjusting a dosage of a medicament administered to a patient to achieve a desired therapeutic effect, preferably while minimizing side effects. For example, a titration regimen may start with a low dose of the medicament being administered to the patient, with subsequent doses slowly increased until an optimal dose is reached or side effects become too pronounced. Titration may find use for medicaments that can affect nerve or hormonal pathways, such as those for high blood pressure, heart disease, seizures, or mood disorders. Titration is also effective for reducing infusion-related reactions that may be caused by patient sensitivity to a new therapy or a therapy's mode of action, as in the case of oncology or endocrinology.

In some instances, a target dose of a medicament may be calculated for a patient. During titration, the patient may be administered a fraction of this target dose at given intervals, with the fraction increasing with each interval until the full target dose is administered. As an example, the patient may be administered 10%, 20%, 30% and then 40% of their target dose at the first four successive intervals, then 100% of the target dose thereafter. Such titration regimens may assist with reducing patient sensitivity to a particular medicament (e.g., reducing the potential for side effects) and/or providing a desired pharmacokinetic/pharmacodynamic (PK/PD) effect (e.g., gradual “loading” of the medicament physiologically over time).

Where the medicament is to be administered to the patient using one or more syringes, the syringes may need to be prepared before administration of the dose. For example, each titrated dose of medicament may need to be drawn from a medicament container such as a medicament vial into a suitable syringe prior to administration of the dose. This process of filling the syringe may be performed for the patient by one or more healthcare professionals such as one or more nurses and/or physicians. However, in some instances, the process of filling the syringe may be performed by the patient themselves, for example in instances where a prefilled (i.e., ready to use or ready to administer) presentation is not available.

Calculation of the titrated doses and preparation of the syringes may be complex and error prone, particularly for patients who may be performing part of the process themselves and for whom titration may be an unfamiliar concept. In some instances, the patient may lose track of which steps of the titration regimen have been completed, may duplicate days (resulting in potential underdoses), or may skip days (resulting in potential overdoses). In some instances, two or more differently-sized syringes may need to be used during the course of a titration regimen. For example, a first dose of medicament may need to be drawn into a first syringe while a second, subsequent dose of medicament may need to be drawn into a second syringe that is smaller or larger than the first syringe. In such instances, the patient may find it difficult to select the correct-sized syringe for a particular dose, and/or they may experience difficulty in drawing the correct doses of medicament into each syringe where the graduations on each syringe differ between syringes.

There may be a desire for improvements in the preparation of syringes, for example the preparation of syringes for medicament titration.

### SUMMARY

According to the present disclosure, there is provided a visual aid for assisting a user in the preparation of a plurality of syringes, the visual aid comprising a plurality of images, wherein each image: is visually representative of a respective syringe of the plurality of syringes; and comprises a respective first visual feature that is representative of a respective first visual feature of the respective syringe and a respective second visual feature that is representative of a respective second visual feature of the respective syringe, the respective first visual feature and the respective second visual feature of the image arranged such that they indicate a respective volume of a medicament to be drawn into the respective syringe.

Each image may be a full-scale image of the respective syringe.

Each image may indicate a position of a respective piston of the respective syringe relative to a respective barrel of the respective syringe, to indicate the respective volume of the medicament to be drawn into the respective syringe.

For each image: the respective first visual feature and the respective second visual feature may be arranged such that a distance between the first visual feature and the second visual feature corresponds to the respective volume of the medicament to be drawn into the respective syringe.

For each image: the respective first visual feature may represent a piston of the respective syringe and the second visual feature may represent at least a portion of a barrel of the syringe.

The visual aid may comprise a sheet material, and wherein the plurality of images are arranged at a front surface of the sheet material.

The sheet material may comprise a plurality of sections arranged linearly across the sheet material, each section containing a respective one of the plurality of images.

The sheet material may comprise a plurality of linear perforations arranged such that each adjacent pair of images has a linear perforation separating the pair of images, wherein the linear perforations are arranged to assist a user in tearing a section of the sheet material from a remaining section of the sheet material.

The visual aid may comprise a clip for releasably coupling a first section of the plurality of sections to a second section of the plurality of sections, to obscure a front surface of a third section of the plurality of sections.

The visual aid may further comprise a sleeve configured to at least partially receive the sheet material such that the sheet material may slide within the sleeve, wherein the sleeve has a window formed therein through which at least one image of the plurality of images is visible.

The plurality of images and the window may be arranged such that the plurality of images are sequentially visible through the window as the sheet material is slid through the sleeve.

The window and the plurality of images may be arranged such that only a single image of the plurality of images may be visible in entirety through the window as the sheet material is slid through the sleeve.

The visual aid may comprise a plurality of sheet materials, each sheet material of the plurality of sheet materials having a respective front surface having a corresponding image of the plurality of images arranged thereon, wherein the plurality of sheet materials are stacked and are releasably coupled together such that each sheet material of the plurality of sheet materials may be uncoupled from the remaining sheet materials.

The visual aid may further comprise a tray for retaining the plurality of syringes, wherein the tray is configured to retain the sheet material such that the plurality of images are visible through the tray.

The tray may comprise a plurality of syringe retainers, each syringe retainer configured to retain a respective syringe of the plurality of syringes, wherein the tray and the sheet material are configured such that each image of the plurality of images is visible through a respective syringe retainer.

Each retainer may comprise a recess configured to at least partially receive a respective syringe of the plurality of syringes, wherein at least a portion of each syringe retainer is substantially transparent such that, when the sheet material is retained in the tray, a respective image of the plurality of images is visible through a respective syringe retainer. The visual aid may further comprise a plurality of dose interval affordances, each dose interval affordance corresponding to a respective one of the plurality of images and comprising an indication of a time that the volume of medicament indicated in the corresponding image should be administered to a patient.

Each image may indicate a size of the respective syringe.

According to a second aspect of the present disclosure, there is provided a system comprising a visual aid disclosed herein and a plurality of syringes.

According to a third aspect of the present disclosure, there is provided a method of using a visual aid as disclosed herein, the method comprising:
using the visual aid to draw a first volume of a medicament into a first syringe of a plurality of syringes.

### BRIEF DESCRIPTION OF THE DRAWINGS

Exemplary embodiments of the present disclosure are described with reference to the accompanying drawings, in which:

FIG. 1A shows a syringe suitable for use with one or more aspects of the present disclosure, prior to being filled with a medicament;

FIG. 1B shows the syringe of FIG. 1A after it has been filled with the medicament;

FIG. 2 shows a visual aid comprising a sheet material according to one or more aspects of the present disclosure, and a plurality of syringes;

FIG. 3 shows an alternative visual aid comprising a sheet material according to one or more other aspects of the present disclosure, and a plurality of syringes;

FIG. 4 shows another alternative visual aid comprising a sheet material according to one or more other aspects of the present disclosure, and a plurality of syringes;

FIG. 5 shows a graphical user interface according to one or more aspects of the present disclosure;

FIG. 6 shows a visual aid comprising a sheet material and a sleeve according to one or more other aspects of the present disclosure;

FIG. 7 shows a visual aid comprising a sheet material having linear perforations according to one or more other aspects of the present disclosure;

FIG. 8 shows a visual aid comprising a plurality of stacked sheet materials according to one or more other aspects of the present disclosure;

FIG. 9 shows a visual aid comprising a sheet material and a tray according to one or more other aspects of the present disclosure, and a plurality of syringes;

FIG. 10 shows the visual aid of FIG. 9 with the sheet material inserted into the tray;

FIG. 11 shows a another visual aid comprising a sheet material according to one or more other aspects of the present disclosure;

FIG. 12 shows the visual aid of FIG. 11 once the sheet material has been folded;

FIG. 13 shows the visual aid of FIG. 12 once the sheet material has been folded at a different location; and

FIG. 14 shows a flowchart illustrating a method according to one or more aspects of the present disclosure.

### DETAILED DESCRIPTION

One or more aspects of the present disclosure relate to visual aids for assisting a user in the preparation of a plurality of syringes. More specifically, one or more visual aids disclosed herein may assist a user in drawing a predetermined volume of medicament into a syringe prior to administration of that medicament, for example as part of a titration regimen. One or more aspects of the present disclosure may assist the user to draw a predetermined volume of medicament into each syringe in a more precise manner. One or more aspects of the present disclosure may assist the user to accurately draw a predetermined volume of medicament into each syringe in a more rapid manner, which may improve the efficiency with which the user may fill the syringes.

Unless otherwise stated, a user as disclosed herein may be a patient to whom the medicament is to be administered, or may be a different person than the patient. The user may be a healthcare professional, such as a nurse or physician, or may not be a healthcare professional (e.g., may be a ‘lay person’).

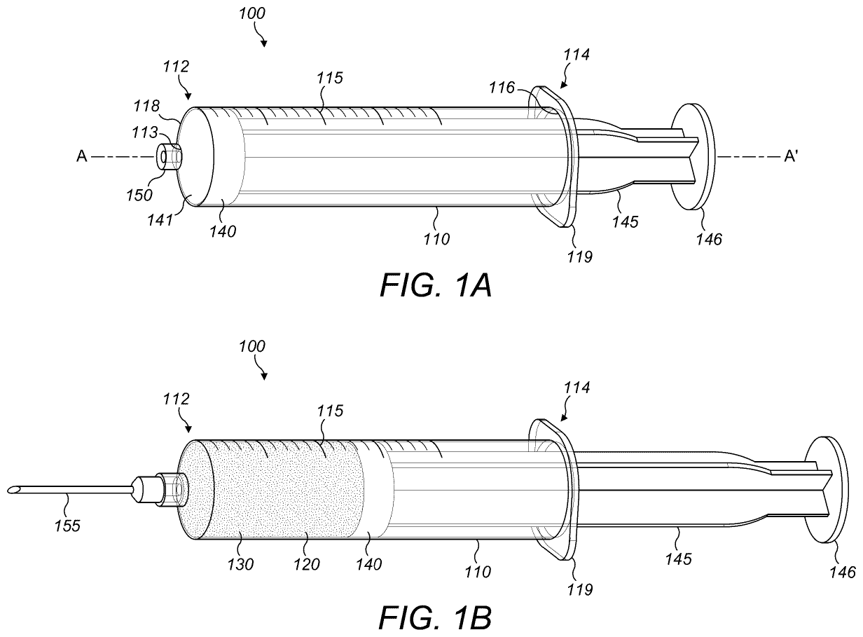

FIGS. 1A and 1B show an example of a syringe 100 that may be suitable for use with one or more aspects of the present disclosure.

The syringe 100 comprises a barrel 110 extending along a longitudinal axis A-A′ of the syringe 100, wherein an interior of the barrel 110 forms a chamber 120 for containing a liquid medicament 130 to be administered (e.g., injected). As shown in FIGS. 1A and 1B, the barrel 110 is substantially cylindrical and has a substantially constant diameter along the longitudinal axis A-A′. The barrel 110 has a distal region 112 and a proximal region 114. The term “distal” refers to a location that is relatively closer to a site of injection, and the term “proximal” refers to a location that is relatively further away from the injection site. The barrel 110 has an open end 116 arranged at the proximal region 114 and a closed end 118 arranged at the distal region 112.

A piston 140 is slidably received within the barrel 110, the piston 140 being moveable along the length of the barrel 110, along the longitudinal axis A-A′, to draw the medicament 130 into the syringe 100 and to expel the medicament 130 from the syringe 100. A circumferential outer surface of the piston 140 is configured to sealingly engage an inner surface of the barrel 110, to inhibit the medicament 130 leaving the chamber 120 via the open end 116 of the barrel 110. The sealing engagement is maintained as the piston 140 is moved distally and proximally within the barrel 110.

A piston rod 145 may be coupled to a proximal end surface of the piston 140 to assist a user in moving the piston 140 relative to the barrel 110. The piston rod 145 may extend substantially along the longitudinal axis A-A′ and have a proximal portion located outside of the barrel 110, proximal of the proximal region 114 of the barrel 110. The piston rod 145 may be permanently coupled to the piston 140 (e.g., by being integrally formed with the piston 140, welded to the piston 140, overmoulded with the piston 140, etc.) or releasably coupled to the piston 140 (e.g., by a releasable threaded engagement between the piston rod 145 and the piston 140, a releasable bayonet coupling, a releasable magnetic coupling, etc.). The piston rod 145 is coupled to the piston 140 such that they move in concert.

To move the piston 140 in a distal direction with respect to the barrel 110, a user may apply a distal force (e.g., distal pushing force) to the piston rod 145 (e.g., to a substantially flat surface of a piston rod flange 146 formed at the proximal portion of the piston rod 145) that causes the piston rod 145 to move in a distal direction with respect to the barrel 110, in turn pushing the piston 140 in the distal direction with respect to the barrel 110. The barrel 110 may have a barrel flange 119 arranged at the proximal region 114 to assist the user in moving the piston rod 145 relative to the barrel 110.

To move the piston 140 in a proximal direction with respect to the barrel 110, a user may apply a proximal force (e.g., proximal pulling force) to the piston rod 145 (e.g., to the piston rod flange 146) that causes the piston rod to move in a proximal direction with respect to the barrel 110, in turn pulling the piston 140 in the proximal direction with respect to the barrel 110. The user may use the barrel flange 119 to assist in moving the piston rod 145 relative to the barrel 110.

The barrel 110 comprises an opening 113 arranged at the distal region 112, the opening 113 arranged to be in fluid communication with the chamber 120 and to act as an outlet and inlet to the chamber 120. The opening 113 is arranged such that medicament 130 contained within the chamber 120 may be expelled out of the chamber 120, via the opening 113, by a movement of the piston 140 in the distal direction relative to the barrel 110. Furthermore, the opening 113 is arranged such that medicament 130 can be drawn into the chamber 120 from an external medicament source such as a medicament vial, via the opening 113, by a movement of the piston 140 in the proximal direction relative to the barrel 110.

As shown in FIG. 1A, the barrel 110 comprises a coupling interface 150 arranged at the opening 113 for coupling a hollow needle 155 or nozzle to the barrel 110 such that, when the hollow needle 155 or nozzle is coupled to the coupling interface 150, the hollow needle 155 or nozzle is in fluid communication with the chamber 120 via the opening 113, allowing medicament 130 to be drawn into the chamber 120 via the hollow needle 155 or nozzle and/or medicament 130 to be expelled from the chamber 120 via the hollow needle 155 or nozzle. FIG. 1B shows the syringe 100 of FIG. 1A after a hollow needle 155 has been coupled to the coupling interface 150. The hollow needle 155 (or nozzle) may be used for drawing the medicament 130 into the chamber 120 (e.g., from a medicament vial) and/or expelling the medicament 130 from the chamber 120 (e.g., during injection into a patient).

In some examples, the coupling interface 150 may be configured to releasably couple the hollow needle 155 or nozzle to the barrel 110 such that the hollow needle 155 or nozzle can be coupled and then uncoupled from the barrel 110 (e.g., to replace the hollow needle 155 or nozzle between uses). In other examples, the coupling interface 150 may be configured to permanently couple the hollow needle 155 or nozzle such that, after the hollow needle 155 or nozzle has been coupled to the barrel 110 using the coupling interface 150, uncoupling of the hollow needle 155 or nozzle by a user is inhibited (e.g., the hollow needle 155 or nozzle cannot be uncoupled without undue effort or destroying a portion of the syringe 100 such as the coupling interface 150). In some instances, the syringe 100 may be provided with a hollow needle 155 or nozzle already coupled to the barrel 110 and in fluid communication with the opening 113 and the chamber 120 (e.g., where the hollow needle 155 or nozzle is integrally formed with the barrel 110).

FIG. 1A shows the syringe 100 in an initial, unfilled stage, wherein the syringe 100 does not yet contain a medicament 130. The piston 140 is arranged at a distal position with respect to the barrel 110 of the syringe 100, at the distal region 112 of the barrel 110.

FIG. 1B shows the syringe of FIG. 1A after a volume of medicament has been drawn into the syringe 100 by a user. In FIG. 1B, the piston 140 is arranged at a position that is proximal with respect to the position of the piston 140 shown in FIG. 1A.

To draw a medicament 130 into the syringe 100, thus filling the chamber 120 with a volume of medicament 130, a user may bring the opening 113 of the syringe 100 into fluid communication an external reservoir of the medicament 130, such as a medicament vial containing the medicament 130. As an example, the user may couple a hollow needle 155, such as a blunt fill needle, to the syringe 100 (if a hollow needle 155 is not already coupled) and insert the hollow needle 155 into a medicament vial such that medicament 130 within the medicament vial may be drawn into the syringe 100 via the hollow needle 155. To draw the medicament 130, the user may translate the piston 140 in a proximal direction relative to the barrel 110 as described previously. Due to the sealing engagement between the piston 140 and the inner surface of the barrel 110, the distal movement of the piston 140 reduces a pressure within a portion of the chamber 120 that is distal to the piston 140, the reduced pressure causing medicament 130 to be drawn from the medicament vial into the chamber 120 via the hollow needle 155 and opening 113.

The user can move the piston 140 proximally relative to the barrel 110 until a desired volume of the medicament 130 has been drawn into the chamber 120, for example as indicated by volume markings 115 provided along the barrel 110. The desired volume may have been calculated prior to drawing the medicament 130 into the syringe 100, and may correspond to a predetermined dose of medicament 130 required for a titration regimen, for example.

To expel the medicament 130 that has been drawn into the syringe 100 as shown in FIG. 1B, for example for administration of the medicament 130 to a patient, the user may translate the piston 140 in a distal direction with respect to the barrel 110, causing a distal end surface 141 of the piston 140 to push the medicament 130 out of the chamber 120, via the outlet 113 and the hollow needle 155, thereby expelling the medicament 130 from the syringe 100. In some examples, prior to expelling the medicament 130, the user may have uncoupled the hollow needle 155 that was used to draw the medicament 130 and coupled a new hollow needle 155 or nozzle to the syringe 100 that is suitable for the particular method of administration of the medicament 130 (e.g., a new hollow needle 155 for an injection, or a nozzle for intra-nasal administration).

Delivery of the medicament 130 into the patient could be sub-cutaneous, intra-muscular, or intravenous, for example, however other routes of medicament delivery may be used instead. Volumes of medicament 130 delivered with such syringes 100 may range from about 0.5 ml to about 2 ml, for example, however this range is not meant to be limiting and it should be understood that in other examples the syringe 100 may be configured to deliver volumes of medicament 130 between about 0.1 ml and about 0.5 ml, or greater than about 2 ml. In some examples, the syringe 100 may be configured to deliver volumes of medicament 130 between about 0.5 ml and about 5.0 ml, between about 0.5 ml and about 10.0 ml, or between about 0.5 ml and about 20.0 ml.

In combination with a specific medicament 130, the presently described syringes 100 may be customized in order to operate within required specifications. For example, the syringes 100 may be customized to inject a medicament 130 within a certain time period (e.g., about 3 to about 20 seconds). Other specifications can include a low or minimal level of discomfort, or to certain conditions related to human factors, shelf-life, expiry, biocompatibility, environmental considerations, etc. Such variations can arise due to various factors, such as, for example, a medicament 130 ranging in viscosity from about 3 cP to about 50 cP. Hollow needles 155 for use with one or more syringes 100 disclosed herein, and for administration of medicament to a patient, could range from about 25 to about 31 Gauge in size, with common sizes being 27 and 29 Gauge. In other instances, such as long acting injectables or depot formations with higher viscosity or shear-thickening characteristics, needles 155 for use with one or more syringes 100 disclosed herein could range from about 16 to about 21 Gauge in size, with common sizes being 19 and 20 Gauge. In any of the foregoing examples, needles 155 may be provided with an outer needle gauge (e.g., 16-31 Gauge) and a thinner inner diameter corresponding to a higher flow needle (e.g., thin wall, extra thin wall, etc.). Such configurations may be advantageously applied to increase the flow rate through the needle and/or reduce manual forces applied to syringe 100 by a user of the apparatus. Needles 155 suitable for use with one or more syringes 100 disclosed herein to draw medicament from a medicament vial or the like could range from about 16 to about 21 Gauge in size, such as from about 16 to about 20 Gauge in size. If the needle 155 is a blunt fill needle, a typical size may be 18 Gauge.

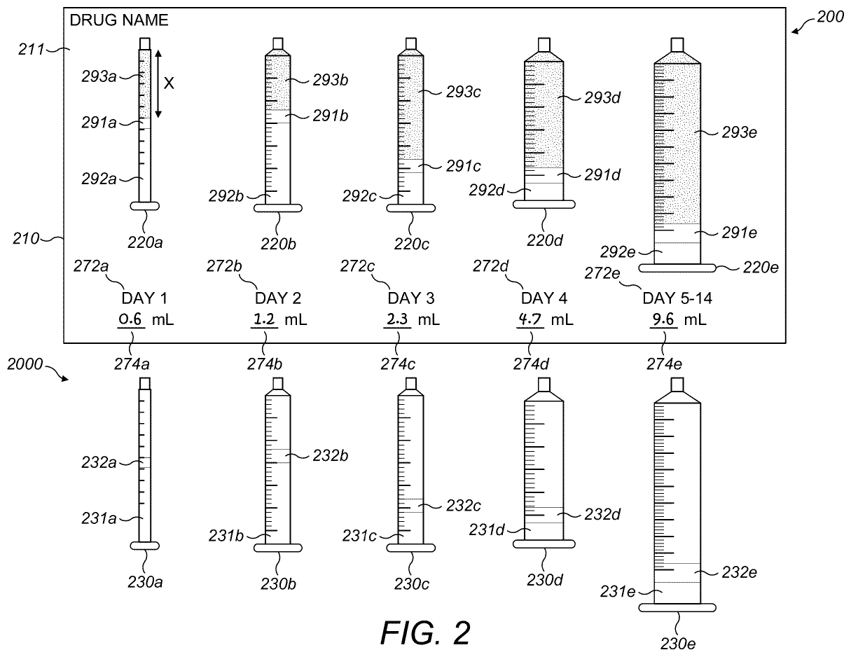

FIG. 2 shows a visual aid 200 in accordance with one or more embodiments of the present disclosure. The visual aid 200 may be for assisting a user in the preparation of a plurality of syringes 230a-230e. In some examples, the plurality of syringes 230a-230e may be provided to the user together with the visual aid 200 as part of a system 2000 (e.g., as part of a kit). Each syringe 230a-230e may be similar or identical to the syringe 100 previously described in relation to FIGS. 1A and 1B.

The visual aid 200 may assist the user in drawing a respective volume of a medicament into each of the plurality of syringes 230a-230e, to fill each syringe 230a-230e. Each volume of medicament to be drawn into each syringe 230a-230e may have been predetermined, for example prior to drawing the volume of medicament into each syringe 230a-230e. In some examples, each volume of medicament may be drawn into each syringe 230a-230e in preparation for administration of the medicament to a patient, for example as part of a titration regimen described previously.

Use of the visual aid 200 may assist the user in drawing the predetermined volume of medicament into each syringe 230a-230e in a more precise manner. Additionally or alternatively, the visual aid 200 may assist the user in accurately drawing a predetermined volume of medicament into each syringe 230a-230e in a more rapid manner, which may improve the efficiency with which the user may fill the syringes 230a-230e. Additionally or alternatively, one or more aspects may assist the user in tracking the correct volume of medicament to be administered at each stage of a multi-dose titration regimen.

As shown in FIG. 2, the visual aid 200 comprises a sheet material 210 having a plurality of images 220a-220e arranged at a front surface 211 of the sheet material 210. The sheet material 210 could be formed from a sheet of card or a sheet of paper, for example, with the plurality of images 220a-220e printed onto the front surface 211. FIG. 2 shows the flat sheet material 210 having a rectangular shape, however it should be understood that this is not meant to be limiting and that in other examples the sheet material 210 may have a different shape than rectangular.

Each image 220a-220e on the sheet material 210 is visually representative of a respective syringe 230a-230e of the plurality of syringes. FIG. 2 shows the plurality of images 220a-220e comprising a first image 220a visually representative of a first syringe 230a, a second image 220b visually representative of a second syringe 230b, a third image 220c visually representative of a third syringe 230c, a fourth image 220d visually representative of a fourth syringe 230d, and a fifth image 220e visually representative of a fifth syringe 230e, with the images 220a-220e spaced linearly across the front surface 211 of the sheet material 210.

Each image 220a-220e may be a full-scale image of the respective syringe 230a-230e it represents. That is, the first image 220a may be a full-scale (i.e., ‘life-sized’) image of a first syringe 230a of the plurality of syringes 230a-230e, the second image 220b may be a full-scale image of a second syringe 230b of the plurality of syringes 230a-130e, the third image 220c may be a full-scale image of a third syringe 230c of the plurality of syringes 230a-230e, the fourth image 220d may be a full-scale image of a fourth syringe 230d of the plurality of syringes 230a-230e, and the fifth image 220e may be a full-scale image of a fifth syringe 230e of the plurality of syringes 230a-230e.

As shown in FIG. 2, each syringe 230a-230e may be substantially similar or identical to the syringe 100 previously described in relation to FIGS. 1A and 1B, with each syringe 230a-230e comprising a respective barrel 231a-231e and a respective piston 232a-232e that may be similar or identical to the barrel 110 and piston 140 described in relation to FIGS. 1A and 1B respectively. In some examples, two or more of the syringes 230a-230e may have different sizes (e.g., different barrel 231a-231e diameters and/or barrel 231a-231e lengths), as shown in FIG. 2.

Each image 220a-220e is configured to assist the user in drawing (filling) a predetermined volume of medicament indicated in each image 220a-220e into the respective syringe 230a-230e also indicated by the image 220a-220e. That is, the first image 220a is configured to assist the user in drawing a respective first volume of the medicament into the first syringe 230a corresponding to a first volume of medicament indicated by the first image 220a, the second image 220b is configured to assist the user in drawing a respective second volume of the medicament into the second syringe 230b corresponding to a second volume of medicament indicated by the second image 220b, the third image 220c is configured to assist the user in drawing a respective third volume of the medicament into the third syringe 230c corresponding to a third volume of medicament indicated by the third image 220c, the fourth image 220d is configured to assist the user in drawing a respective fourth volume of the medicament into the fourth syringe 230d corresponding to a fourth volume of medicament indicated by the fourth image 220d, and the fifth image 220e is configured to assist the user in drawing a respective fifth volume of the medicament into the fifth syringe 230e corresponding to a fifth volume of medicament indicated by the fifth image 220d.

To assist the user in drawing the respective volumes of medicament, each image 220a-220e comprises at least a respective first visual feature 291a-291e and a respective second visual feature 292a-292e. For each image 220a-220e, the first visual feature 291a-291e is representative of a corresponding first visual feature of the respective syringe 230a-230e and the respective second visual feature 242a-242e is representative of a respective second visual feature of the respective syringe 230a-230e.

For each image 220a-220e, the respective first visual feature 291a-291e and the respective second visual feature 292a-292e are arranged relative to each other such that they indicate to the user the respective volume of the medicament to be drawn into the respective syringe 230a-230e. For example, for each image 220a-220e, the respective first visual feature 291a-291e and the respective second visual feature 292a-292e are arranged such that a distance (e.g., distance X) between the first visual feature 291a-291e and the second visual feature 292a-292e corresponds to the respective volume of the medicament to be drawn into the respective syringe 230a-230e.

In the example shown in FIG. 2, the respective first visual feature 291a-291e comprises an image of a respective piston that represents a corresponding piston 232a-232e of the respective syringe 230a-230e and the second visual feature 292a-292e comprises an image of a respective barrel that represents at least a corresponding portion of a barrel 231a-231e of the respective syringe 230a-230e. FIG. 2 further shows each image 220a-220e comprising a third visual feature 293a-293e, the third visual feature 293a-293e comprising an image of a volume of the medicament that is to be drawn into the respective syringe 230a-230e by a user.

Each image 220a-220e therefore indicates a position of a respective piston 232a-232e of the respective syringe 230a-230e relative to a respective barrel 231a-231e of the respective syringe 230a-230e, to indicate the respective predetermined volume of the medicament to be drawn into the respective syringe 230a-230e.

FIG. 2 shows the visual aid 200 further comprising additional information arranged at the front surface 211 of the sheet material 210 to assist the user in preparing and/or administering the volumes of medicament. Specifically, FIG. 2, shows a plurality of dose interval affordances 272a-272e and a plurality of dose volume affordances 272a-274e arranged at the front surface 211. Each dose interval affordance 272a-272e and each dose volume affordance 272a-274e corresponds to, and is arranged adjacent, as respective one of the images 220a-220e. Each dose interval affordance 272a-272e comprises an indication of a time (e.g., hour, day, week or month) that the volume of medicament indicated in the corresponding image 220a-220e should be administered to the patient. Each dose volume affordance 274a-274e comprises a text indication of the volume of medicament that is indicated in the corresponding image 220a-220e.

Each image 220a-220e may also indicate a respective size of the respective syringe 230a-230e. For example, a width and/or length of the image 220a-220e may be the same as the corresponding width and/or length of the corresponding syringe 230a-230e.

To use the visual aid 200, the user views the first image 220a on the sheet material 210 and draws a first volume of a medicament into the first syringe 230a (e.g., in a similar manner as previously described in relation to FIGS. 1A and 1B) using the first image 220a that corresponds to the volume of medicament indicated by the first visual feature 291a and second visual feature 292a of the first image 220a. During or after the drawing process, the user may hold the first syringe 230a adjacent the first image 220a to ensure the volume of medicament drawn into the first syringe 230a is equal to the volume of medicament indicated by the first image 220a. Once the correct, predetermined volume of medicament has been drawn into the first syringe 230a, the first syringe 230a may be used to administer the medicament to a patient.

The user may subsequently fill one or more of the remaining syringes 230b-230e with their respective volumes of medicament as indicated by their respective images 220b-220e, in a similar manner as filling the first syringe 230a.

In some instances, the user may fill the one or more remaining syringes 230b-230e immediately after filling the first syringe 230a. In some instances, the user may fill the one or more remaining syringes 230b-230e in accordance with a schedule (e.g., in accordance with a titration schedule).

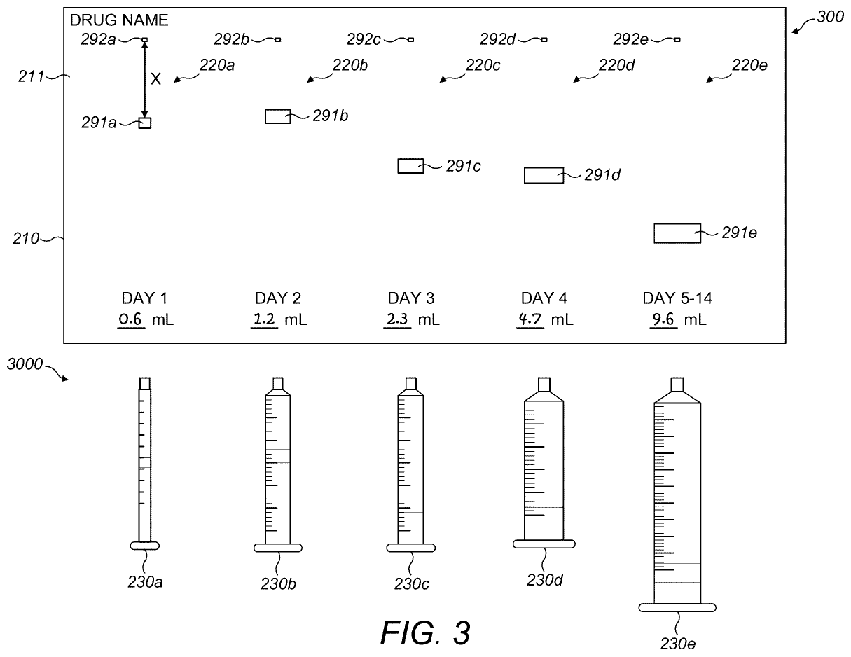

FIG. 3 shows a visual aid 300 in accordance with one or more further embodiments of the present disclosure.

The visual aid 300 may be similar or identical to the visual aid 200 described in relation to FIG. 2, comprising a sheet material 210 having a plurality of images 220a-220e arranged at a front surface 211 of the sheet material 210 as previously described in relation to FIG. 2. However, the images 220a-220e of the visual aid 300 differ to the visual images 220a-220e of the visual aid 200.

As shown in FIG. 3, each image 220a-220e of the visual aid 300 is visually representative of a respective syringe 230a-230e of a plurality of syringes that may be supplied with the visual aid 300 as part of a system 3000 (e.g., kit). However, each image 220a-220e of the visual aid 300 takes a more schematic form compared to the images 220a-220e of the visual aid 200 shown in FIG. 2.

As shown in FIG. 3, the respective first visual feature 291a-291e comprises an image of a respective piston that represents a corresponding piston of the respective syringe 230a-230e and the second visual feature 292a-292e comprises an image of a portion of barrel (in this case, a distal end of a barrel) that represents at least a corresponding portion (i.e., distal end) of a barrel of the respective syringe 230a-230e.

The visual aid 300 may be used in a similar manner to the visual aid 200.

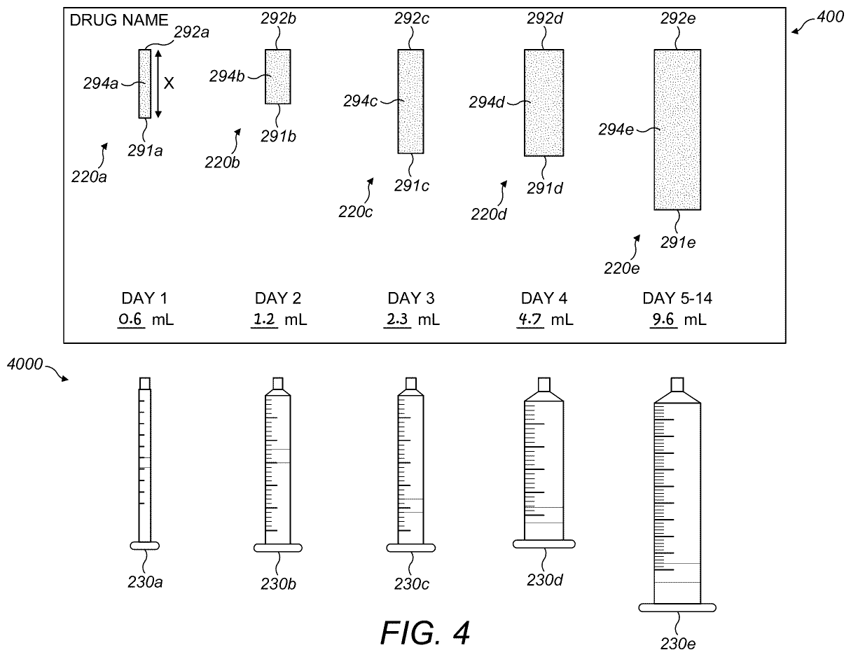

FIG. 4 shows a visual aid 400 in accordance with one or more further embodiments of the present disclosure.

The visual aid 400 may be similar or identical to the visual aid 200 described in relation to FIG. 2, comprising a sheet material 210 having a plurality of images 220a-220e arranged at a front surface 211 of the sheet material 210 as previously described in relation to FIG. 2. However, the images 220a-220e of the visual aid 400 differ to the visual images 220a-220e of the visual aid 200.

As shown in FIG. 4, each image 220a-220e of the visual aid 400 is visually representative of a respective syringe 230a-230e of a plurality of syringes that may be supplied with the visual aid 400 as part of a system 400 (e.g., kit). However, each image 220a-220e of the visual aid 400 takes a more schematic form compared to the images 220a-220e of the visual aid 200 shown in FIG. 2.

As shown in FIG. 4, each image 220a-220e comprises a respective image 294a-294e of the volume of medicament to be drawn into the respective syringe 230a-230e. In this case, the respective first visual feature 291a-291e comprises a first end of the image 294a-294e of the volume of medicament (which corresponds to a distal end surface of a piston of the respective syringe 230a-230e) and the second visual feature 292a-292e a second end of the image 294a-294e of the volume of medicament (which corresponds to a distal end of a barrel of the respective syringe 230a-230e) that is opposite the first end.

The visual aid 400 may be used in a similar manner to the visual aid 200.

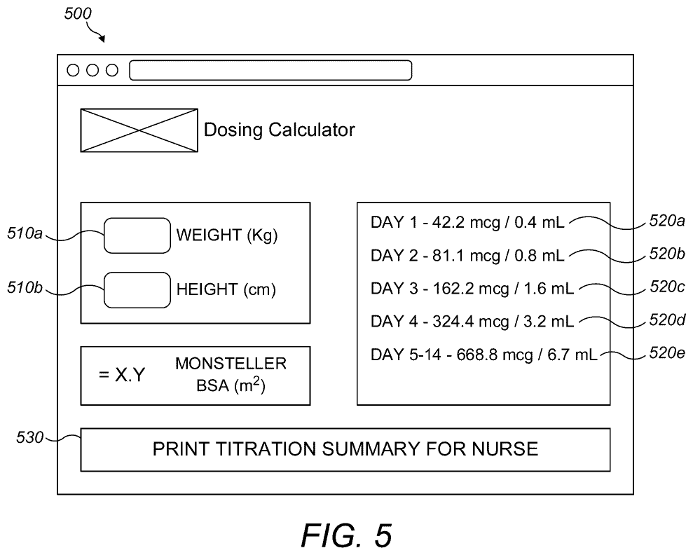

For one or more embodiments disclosed herein, the volumes of medicament to be drawn into each syringe 230a-230e may have been generated using computer-implemented software. For example, FIG. 5 shows an example of a graphical user interface (GUI) 500 that may be used to determine the predetermined volumes of medicament to be drawn into each syringe 230a-230e. The GUI comprises one or more input elements 510a-510b into which information (e.g., height, weight, etc.) corresponding to a patient may be input. The software (using one or more processers) may process the information to determine the plurality of volumes 520a-520e of medicament, which may be output for display at the GUI 500. In some examples, a user-selectable input element 530 may be provided at the GUI 500, wherein user selection of the user selectable input element 530 (e.g., via user selection on a touchscreen, using a pointer such as a mouse, or using a keyboard, etc.) may cause a sheet material (e.g., sheet material 210) as disclosed herein to be printed (e.g., using a printer) with images 220a-220e printed onto the sheet material corresponding to the volumes 520a-520e of medicament determined by the software.

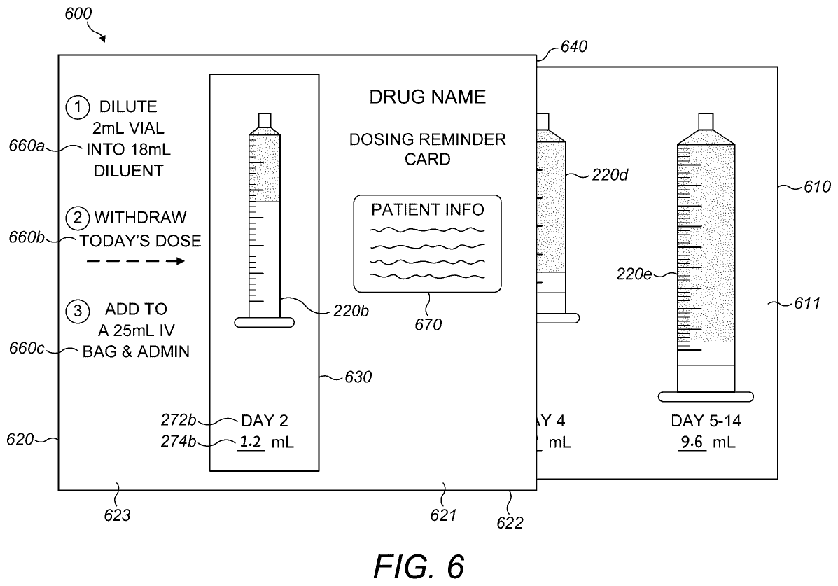

FIG. 6 shows a visual aid 600 in accordance with one or more further embodiments of the present disclosure.

As shown in FIG. 6, the visual aid 600 comprises a sheet material 610 which may be similar or identical to the sheet material 210 previously described in relation to FIG. 2, the sheet material 610 having a plurality of images 220a-220e arranged at a front surface 611 of the sheet material 610 in a similar or identical manner as previously described in relation to FIG. 2.

However, FIG. 6 shows the visual aid 600 further comprising a sleeve 620. The sleeve 620 is configured to at least partially receive the sheet material 610 such that the sheet material 610 may slide within the sleeve 620. The sleeve 620 is substantially flat and comprises a substantially flat front panel 621 and a substantially flat rear panel 622 arranged such that the sheet material 610 is slidingly receivable between the front panel 621 and the rear panel 622 via a slot 640 formed by the front panel 621 and rear panel 622. The sleeve 620 comprises a slot 640 arranged between the front panel 621 and the rear panel 622, through which the sheet material 610 may be received, such that the sheet material 610 is slidingly receivable within the sleeve 620.

The sleeve 620 has a window 630 formed in a front surface 623 of the front panel 621 sleeve 620 through which at least one image 220a-220e of the plurality of images is visible to a user when the sheet material 610 is received in the sleeve 620. For example, FIG. 6 shows the second image 220b visible through the window 630. At least the first image 220a and the third image 220c are hidden from view by the front panel 621 of the sleeve 620.

To use the visual aid 600, a user may insert the sheet material 610 into the sleeve 620 via the slot 640 and move (e.g., translate) the sheet material 610 relative to the sleeve 620. As the sheet material 610 moves relative to the sleeve 620, each of the images are sequentially visible through the window 630. As shown in FIG. 6, the window 630 and the plurality of images 220a-220e may be arranged such that only a single image 220a-220e of the plurality of images may be visible in entirety through the window 630 at any given time as the sheet material 610 is slid through the sleeve 620.

The user may initially position the sheet material 610 relative to the sleeve 620 such that the first image 220a is visible through the window 630. The user may then use the first image 220 to draw a first dose of medicament into a first syringe (e.g., first syringe 230a), for example in a similar manner as described in relation to FIG. 2. The first dose may be administered to a patient.

At a later point in time, when a second dose of medicament is to be administered to the patient, the user may move (e.g., slide) the sheet material 610 relative to the sleeve 620 such that the second image 220b is now visible through the window 630 (and the first image 220a may now no longer be visible through the window 630). The user may then use the second image 220b to draw a second dose of medicament into a second syringe (e.g., second syringe 230b), for example in a similar manner as described in relation to FIG. 2. The second dose may be administered to the patient.

A similar process of sliding the sheet material 610 relative to the sleeve 620 may be performed for preparing any subsequent doses of medicament (e.g., a third dose, fourth dose, fifth dose, . . . etc.).

In some examples, one or more preparation instructions 660a-660c may be provided on the sleeve 620, the preparation instructions 660a-660c providing textual instructions to assist the user in preparing the syringes 230a-230e. For example, the one or more preparation instructions 660a-660c may comprise instructions to assist the user in mixing the medicament and/or drawing the medicament into the syringes 230a-230e.

In some embodiments, patient information 670 may be provided on the sleeve 620, wherein the patient information 670 may comprise information regarding the specific patient to whom the medicament is to be administered, for example to aid the user in preparing and/or administering the medicament. In some embodiments, a portion of information corresponding to the medicament is prepopulated during manufacture of the medicament, while in other embodiments, a portion of patient information 670 is applied to the apparatus during the dispensing by a pharmacy, or combinations thereof.

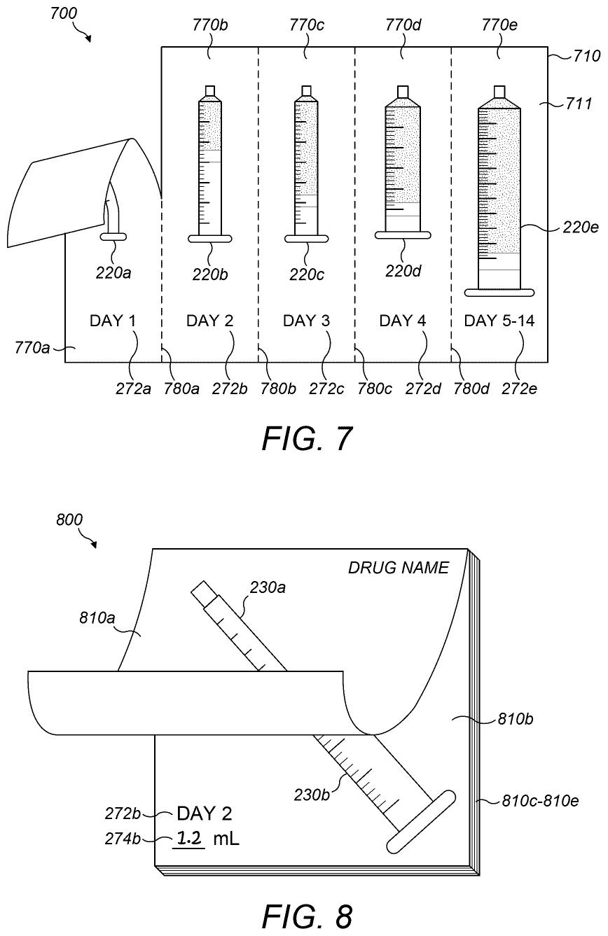

FIG. 7 shows a visual aid 700 in accordance with one or more embodiments of the present disclosure. The visual aid 700 may be substantially similar to the visual aid 200 previously described in relation to FIG. 2, comprising a sheet material 710 which may be similar or identical to the sheet material 210 previously described in relation to FIG. 2, the sheet material 710 having a plurality of images 220a-220e arranged at a front surface 711 of the sheet material 710 in a similar manner as previously described in relation to FIG. 2.

However, the sheet material 710 further comprises a plurality of substantially linear perforations 780a-780d.

Each linear perforation 780a-780d extends from one edge of the sheet material 710 to an opposite edge of the sheet material 710. The linear perforations 780a-780d divide the sheet material 710 into a plurality of sections 770a-770e, with each section 770a-770e having a respective one of the images 220a-220e formed thereon. The linear perforations 780a-780d are therefore arranged such that each adjacent pair of images 220a-220e has a linear perforation 780a-780d separating that pair of images 220a-220e. The linear perforations 780a-780d are arranged to assist a user in tearing a section of the sheet material away from a remaining section of the sheet material 710.

The visual aid 700 may initially be provided to a user with all linear perforations 750a-760d intact, such that each of the sections 770a-770e of the sheet material 710 remained joined to each other. In use, a user may use the first image 220a to draw a first volume of medicament into a first syringe 230a, for example as previously described in relation to FIG. 2. The first volume may be administered to a patient. After drawing the first volume of medicament, the user may separate the first section 770a of the sheet material 710 from the remaining second to fifth sections 770b-770e of the sheet material 710 by tearing along a first linear perforation 780a. FIG. 7 shows the first section 770a half-torn from the second section 770b. In some instances, the user may discard the first section 770a after it has been fully separated from the second section 770b. Removing the first section 770a may serve as a reminder to the user that the next dose of medicament should correspond to the volume indicated in the second image 220b. The user may then prepare a second volume of medicament using the second image 220b, in a similar manner to the first volume of medicament, and separate the second section 770b of the sheet material 710 from the third section 770c of the sheet material 710 by tearing along the second linear perforation 780b. A similar process may be repeated for each remaining volume of medicament to be administered (e.g., third volume, fourth volume, fifth volume, etc.).

FIG. 8 shows a visual aid 800 in accordance with one or more further embodiments of the present disclosure. The visual aid 800 may be substantially similar to the visual aid 200 previously described in relation to FIG. 2, comprising a plurality of images 220a-220e as previously described in relation to FIG. 2. However, rather than comprising a single sheet material 210, the visual aid 800 comprises plurality of sheet materials, each sheet material 810a-810e of the plurality of sheet materials having a respective front surface 811a-811e having a corresponding image 220a-220e of the plurality of images arranged thereon. As shown in FIG. 8, the plurality of sheet materials 810a-810e are stacked and are releasably coupled together by adhesive portions such that each sheet material 810a-810e of the plurality of sheet materials may be uncoupled from the remaining sheet materials.

In use, a user may use the first image 220a to draw a first volume of medicament into a first syringe (e.g., first syringe 230a), for example in a similar manner as previously described in relation to FIG. 2. The user may then uncouple the first sheet material 810a on which the first image 220a is provided from the second sheet material 810b arranged beneath. The user may then use the second image 220b provided on the second sheet material 810b to draw a second volume of medicament into a second syringe (e.g., second syringe 230b). The user may then uncouple the second sheet material 810b on which the second image 220b is provided from the third sheet material 810c arranged beneath. This process may be repeated for subsequent doses of medicament. The provision of plurality of sheet materials 810a-810e that are stacked and are releasably coupled together may allow a user to easily keep track of the next dose of medicament to be prepared and/or administered.

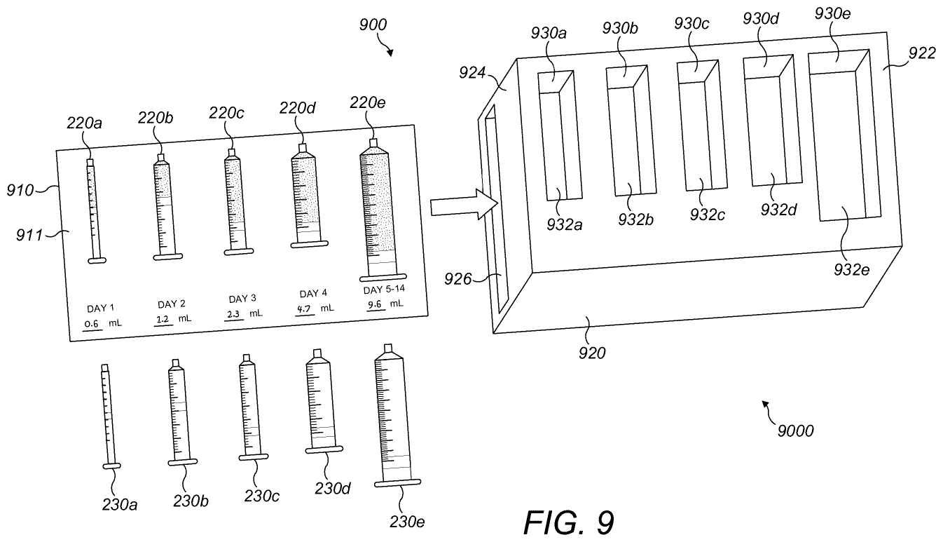

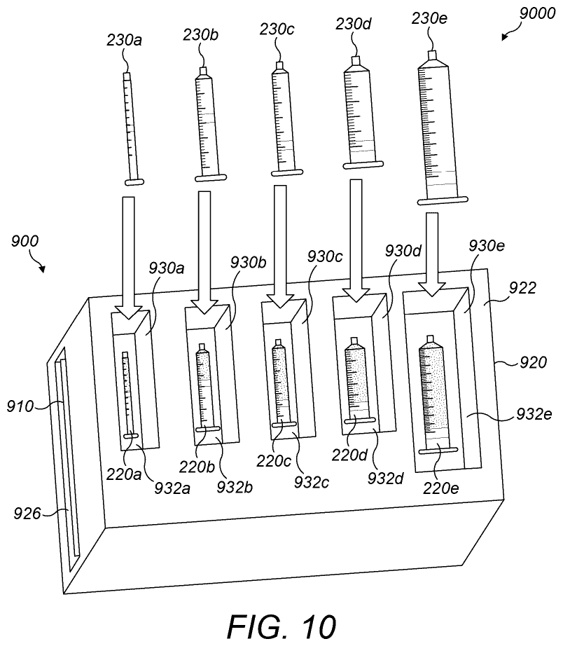

FIGS. 9 and 10 show a visual aid 900 in accordance with one or more embodiments of the present disclosure.

As shown in FIG. 9, the visual aid 900 comprises a sheet material 910 which may be similar or identical to the sheet material 210 previously described in relation to FIG. 2, the sheet material 310 previously described in relation to FIG. 3, or the sheet material 410 previously described in relation to FIG. 4, for example. The sheet material 910 has a plurality of images 220a-220e arranged at a front surface 911 of the sheet material 910, in a similar manner as previously described in relation to FIG. 2, for example.

However, FIG. 9 shows the visual aid 900 further comprising a tray 920 that acts as a packaging for retaining a plurality of syringes 230a-230e. The plurality of syringes 230a-230e may be provided with the visual aid 900 as part of a system 9000 (e.g., kit)

The tray 920 comprises a plurality of syringe retainers 930a-930e arranged at an upper surface 922 of the tray 920, each syringe retainer 930a-930e configured to retain a respective syringe 230a-230e of the plurality of syringes. FIG. 9 shows each syringe retainer 930a-930e comprising a corresponding recess 932a-932e configured to at least partially receive a respective syringe 230a-230e. That is, each recess 932a-932e may have a shape that can at least partially receive a respective syringe 230a-230e. FIG. 9 shows a first syringe retainer 930a configured to receive the first syringe 230a, a second syringe retainer 930b configured to receive the second syringe 230b, a third syringe retainer 930c configured to receive the third syringe 230c, a fourth syringe retainer 930d configured to receive the fourth syringe 230d, and a fifth syringe retainer 930e configured to receive the fifth syringe 230e.

In some examples, each syringe retainer 930a-930e is configured to receive a specific size of each respective syringe 230a-230e. In some examples, each syringe retainer 930a-930e is separately configured to receive a range of syringe sizes for each respective syringe 230a-230e, where the range of syringe sizes corresponds to a minimum and a maximum dose volume for each preparation (e.g., titration step). In some examples, each syringe retainer 930a-930e is configured to retain a specific syringe size and inhibit insertion of one or more other syringe sizes, providing a user of the apparatus a “forcing function” preventing inappropriate use of the tray 920. In some examples, each syringe retainer 930a-930e is configured to fit a specific prefilled syringe 230a-230e, including specific user affordances (e.g., add-on finger flanges), where each syringe 230a-230e corresponds to a specific dose or device in a predetermined sequence.

The tray 920 is configured to retain the sheet material 910 such that the plurality of images 220a-220e are visible through the tray 920 when the sheet material 910 is retained in the tray 920. For example, a side wall 924 of the tray 920 may comprise a slot 926 through which the sheet material 910 may be inserted into a cavity within the tray 920, to retain the sheet material 910 in the tray. FIG. 9 shows the sheet material 910 prior to insertion into the tray, while FIG. 10 shows the sheet material 910 after it has been inserted into, and retained within, the tray 920, via the slot 926.

The tray 920 and the sheet material 910 are configured such that each image 220a-220e of the plurality of images is visible through a respective syringe retainer 930a-930e when the sheet material 910 is retained within the tray 920, as shown in FIG. 10. At least a portion of each syringe retainer 930a-930e (e.g. a bottom surface of each recess 932a-932e) is substantially transparent such that, when the sheet material 910 is retained in the tray 920, a respective image 220a-220e of the plurality of images is visible through a respective syringe retainer 930a-930e, via the substantially transparent portion. For example, the substantially transparent portions of each syringe retainer 930a-930e may be formed from a substantially transparent polymer. In some instances, the entire tray 920 may be formed from a substantially transparent polymer.

FIG. 10 shows that each image 220a-220e is aligned with a respective syringe retainer 930a-930e (e.g., aligned with a respective recess 932a-932e) when the sheet material 910 is retained in the tray 920.

Having each image 220a-220e visible through a respective syringe retainer 930a-930e may be beneficial when assembling the system 9000, for example prior to delivery to a user. For example, to assemble the system 9000, a user (who may be different or the same as the end user) may insert the sheet material 910 into the tray 920 via the slot 926 such that each image 220a-220e is aligned with, and visible through, a respective syringe retainer 930a-930e of the tray 920. The user may then insert each syringe 230a-230e into a respective syringe retainer 930a-930e. Since each image 220a-220e is representative of a particular syringe 230a-230e of the plurality of syringes, the visibility of the images 220a-220e through the syringe retainers 930a-930e may assist the user in placing the correct syringe 230a-230e into the correct corresponding syringe retainer 930a-930e.

Once all syringes 230a-230e have been retained in their respective syringe retainers 930a-930e, the assembled system 9000 may then be used to draw volumes of medicament into each syringe 230a-230. For example, a user (e.g., end user) may use the images 220a-220e on the sheet material 910 to draw the correct predetermined volumes of medicament into the correct syringes 230a-230e, for example in a similar manner as previously described in relation to FIGS. 1A and 1B. In some examples, the sheet material 910 may remain retained in the tray 920 as the user draws the volumes of medicament. In other examples, the user may remove the sheet material 910 from the tray 920 via the slot 926 prior to using the sheet material 910 to draw the volumes of medicament.

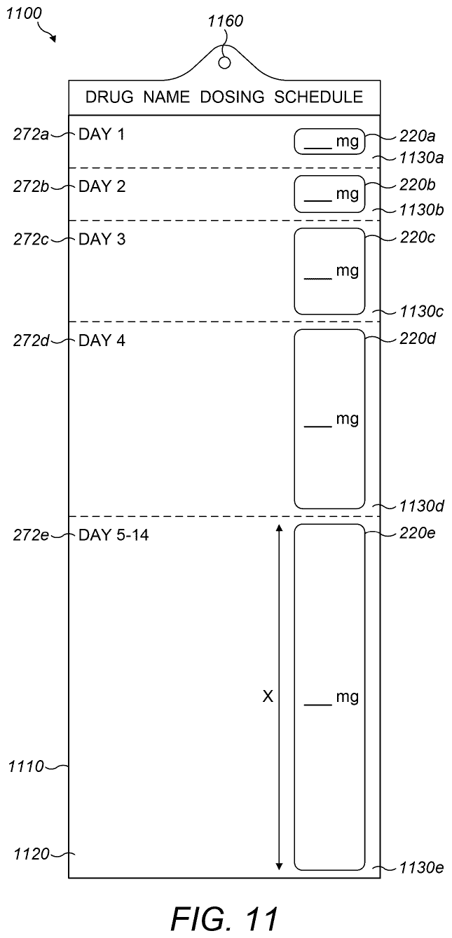

FIGS. 11 to 13 shows a visual aid 1100 according to one or more further embodiments of the present disclosure.

As shown in FIG. 11, the visual aid 800 comprises a sheet material 1110 which may be similar to the sheet material 210 previously described in relation to FIG. 2, the sheet material 1100 having a plurality of images 220a-220e arranged at a front surface 1120 of the sheet material 810 in a similar manner as previously described in relation to FIG. 2.

As shown in FIG. 11, the sheet material 1110 comprises a plurality of sections 1130a-1130e arranged linearly down front surface 1120 the sheet material 1110, each section 1130a-1130e containing a respective one of the plurality of images 220a-220e. A hanging arrangement 1160 may be arranged at an end of the sheet material 1110 (e.g., adjacent the first section 1130a) for hanging the sheet material 1110. For example, the hanging arrangement 1160 may comprise an aperture for receiving a projection (e.g., a nail, pin or screw) to hang the sheet material 1110 on a wall. The sheet material 1110 may be arranged such that, when hung on a wall using the hanging arrangement 1160, the sections 1130a-1130e are arranged sequentially in a direction vertically down from the hanging arrangement 1160.

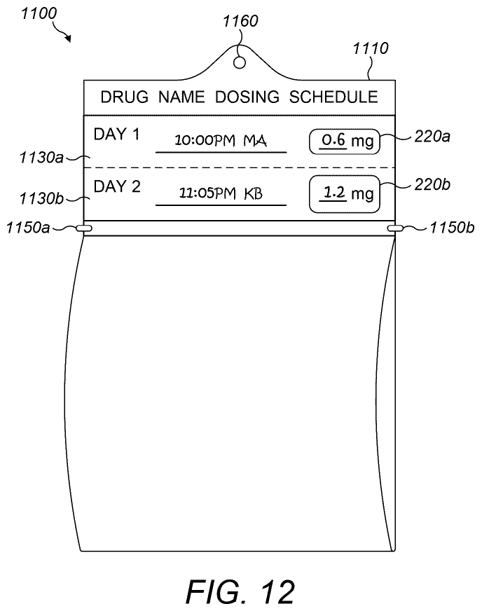

FIG. 11 shows the visual aid 1100 prior to use. The visual aid 1100 comprises at least one clip 1150a-b for releasably coupling one section of the plurality of sections 1130a-1130e to a different section of the plurality of sections 1130a-1130e, to obscure yet another section of the plurality of sections 1130a-1130e, as shown in FIG. 12.

To prepare the visual aid 1100 for use, a user may fold the sheet material 1110 such that first image 220a on the first section 1130a is visible but the second to fifth images 220b-220e are hidden within the folded portion of the sheet material 1110, and use the one or more clips 1150a, 1150b (or another coupling element) to couple the folded sections of the sheet material 1110 together to hold them in place. A user may then use the first image 220a to draw a first volume of medicament into a first syringe (e.g., first syringe 230a), for example as described previously.

After the first volume of medicament has been drawn into the first syringe, the user may refold the sheet material 1110 at a new position such that the second image 220b on the second section 1130b is now visible but the third to fifth images 220c-220e remain hidden within the folded portion of the sheet material 1110. The user may use the one or more clips 1150a, 1150b (or another coupling element) to couple the folded sections of the sheet material 1110 together to hold them in this new position, as shown in FIG. 12. The user may then use the second image 220b to draw a second volume of medicament into a second syringe (e.g., second syringe 230b), for example as described previously.

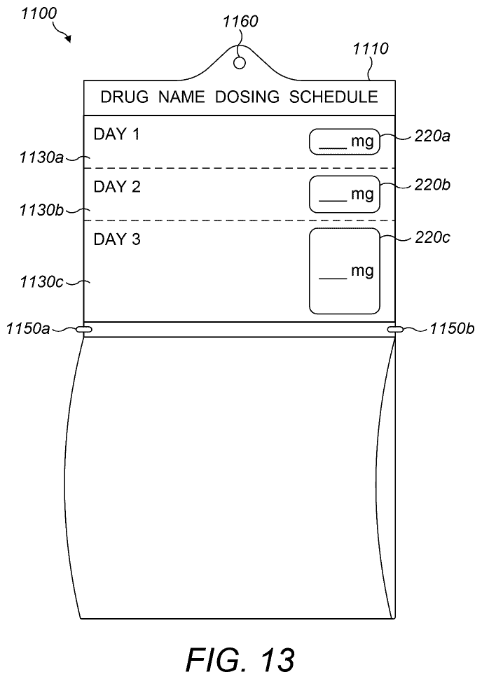

After the second volume of medicament has been drawn into the first syringe, the user may refold the sheet material 1110 at a new position such that the third image 220c on the third section 1130c is now visible but the fourth and fifth images 220d, 220e remain hidden within the folded portion of the sheet material 1110. The user may use the one or more clips 1150a, 1150b (or another coupling element) to couple the folded sections of the sheet material 1110 together to hold them in this new position, as shown in FIG. 13. The user may then use the third image 220c to draw a third volume of medicament into a third syringe (e.g., third syringe 230c), for example as described previously.

The user may repeat the above process for any remaining volumes of medicament to be drawn sequentially uncovering a new section 1130a-1130e of the sheet material 1110.

By allowing a user to cover and then sequentially uncover the plurality of images 220a-220e, the visual aid 1100 may assist the user in keeping track of the next volume of medicament to be drawn and/or administered.

In some examples, each image 220a-e may be provided with a portion in which the user or a third party (e.g., pharmacist) may write in information related to the dose of medicament corresponding to that image 220a-e (e.g., the volume of the dose).

In some examples, the distance X of each image 220a-e may double between each image 220a-e in sequence (e.g., the distance X for image 220b may be double the distance X for image 220a, the distance X for image 220c may be double the distance X for image 220b, the distance X for image 220d may be double the distance X for image 220c, and the distance X for image 220e may be double the distance X for image 220d). This may be beneficial in communicating to the user in a simple manner that the dose of medicament is to be doubled each time.

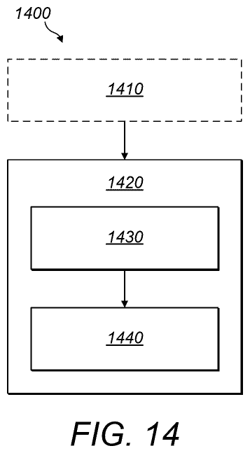

FIG. 14 shows a flowchart illustrating a method 1400 according to one or more aspects of the present disclosure. The method 1400 may be a method of using any visual aid disclosed herein (e.g., visual aid 200, 300, 400, 500, 600, 700, 800, 900, or 1100).

At an optional step 1410, the visual aid may be provided to a user.

At step 1420, the visual aid is used to draw a first volume of a medicament into a first syringe of a plurality of syringes (e.g., a first syringe 230a).

Step 1420 may comprise a first sub step 1430, at which the user draws the first volume of the medicament into the first syringe.

Step 1420 may further comprise a second sub step 1440, at which the user compares (using the visual aid) the first volume of medicament drawn into the first syringe with the volume of medicament indicated by a first image (e.g., first image 220a) of the visual aid. Based on the comparison, the user may decide whether further medicament should be drawn into the first syringe, or whether a portion of the medicament drawn into the first syringe should be expelled, to match the volume of medicament indicated by the first image. The user may draw and/or expel medicament into/out of the first syringe until the volume of medicament within the first syringe corresponds to the volume of medicament indicated by the first image.

In some instances, sub step 1440 may be performed after sub step 1430. In other instances, sub step 1430 and sub step 1440 may be performed substantially simultaneously.

Once the user has drawn a volume of medicament into the first syringe that corresponds to the volume of medicament indicated by the first image, the user may perform step 1420 on one or more other syringes (e.g., syringes 230b-230e) based on the images (e.g., images 220b-220e) corresponding to those syringes.

While one or more aspects of the present disclosure have been described in the context of titration, it should be understood that this is not meant to be limiting, and that one or more aspects of the present disclosure may be implemented as part of a medicament administration procedure that does not involve titration.

While one or more aspects of the present disclosure have been described in the context of five images 220a-220e corresponding to five syringes 230a-230e, it should be understood that this is not meant to be limiting and that one or more of the visual aids disclosed herein may comprise two images 220a-220b, three images 220a-220c, four images 220a-220d, or six or more images, with the number of corresponding syringes 230a-230e being equal to the number of images 220a-220e.

While one or more aspects of the present disclosure show the syringes 230a-230e without respective piston rods (e.g., similar or identical to piston rod 145), it should be understood that this is not meant to be limiting and that in some examples one or more of the syringes 230a-230e disclosed herein may each comprise a respective piston rod. Similarly, while one or more aspects of the present disclosure show the images 220a-220e without piston rods (e.g., similar or identical to piston rod 145), it should be understood that this is not meant to be limiting and that in some examples one or more of the images 220a-220e disclosed herein may each show a respective piston rod.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255,Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen.

The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen.

Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Those of skill in the art will understand that modifications (additions and/or removals) of various components of the embodiments described herein may be made without departing from the full scope and spirit of the present invention, which encompass such modifications and any and all equivalents thereof.

### LIST OF REFERENCE NUMBERS

100—syringe
110—barrel
112—distal region (of barrel)
113—opening
114—proximal region (of barrel)
115—volume markings
116—open end (of barrel)
118—closed end (of barrel)
119—barrel flange
120—chamber
130—medicament
140—piston
141—distal end surface (of piston)
145—piston rod
146—piston rod flange
150—coupling interface
155—hollow needle
200—visual aid
210—sheet material
211—front surface (of sheet material)
220a-e—images
230a-e—syringes
231a-e—barrel
232a-e—piston
272a-e—dose interval affordance
274a-e—dose volume affordance
291a-e—first visual feature
292a-e—second visual feature
293a-e—third visual feature
294a-e—image of volume of medicament
300—visual aid
400—visual aid
500—graphical user interface
510a-b—input elements
520a-e—volumes of medicament
530—user-selectable input element 530
600—visual aid
610—sheet material
611—front surface
620—sleeve
621—front panel
622—rear panel
630—window
640—slot
660a-c—preparation instructions
670—patient information
700—visual aid
770a-e—sections
780a-d—linear perforations
800—visual aid
810a-810e—sheet materials
811a-811e—front surfaces
900—visual aid
910—sheet material
911—front surface
920—tray
922—upper surface
924—side wall
926—slot
930a-e—syringe retainer
932a-e—recess
1100—visual aid
1110—sheet material
1120—front surface
1130a-e—sections
1150a-b—clips
1160—hanging arrangement
1400—method
1410—first method step
1420—second method step
1430—first method sub step
1440—second method sub step
2000—system
3000—system
4000—system

1-20. (canceled)

21. A visual aid for assisting a user in the preparation of a plurality of syringes, the visual aid comprising:
a sheet material comprising a plurality of sections arranged linearly across the sheet material, the sheet material comprising a plurality of images arranged on a front surface of the sheet material, wherein each section of the plurality of sections contains an image of the plurality of images,
wherein each image:
is visually representative of a respective syringe of the plurality of syringes; and
comprises a respective first visual feature that is representative of a respective first visual feature of the respective syringe and a respective second visual feature that is representative of a respective second visual feature of the respective syringe, the respective first visual feature and the respective second visual feature of the image arranged such that they indicate a respective volume of a medicament to be drawn into the respective syringe, wherein each respective volume of medicament represents a given dose in a singular prescribed treatment.

22. The visual aid of claim 21, wherein each image is a full-scale image of the respective syringe.

23. The visual aid of claim 21, wherein each image indicates a position of a respective piston of the respective syringe relative to a respective barrel of the respective syringe, to indicate the respective volume of the medicament to be drawn into the respective syringe.

24. The visual aid according to claim 21, wherein, for each image:
the respective first visual feature and the respective second visual feature are arranged such that a distance between the first visual feature and the second visual feature corresponds to the respective volume of the medicament to be drawn into the respective syringe.

25. The visual aid according to claim 21, wherein, for each image:
the respective first visual feature represents a piston of the respective syringe and the second visual feature represents at least a portion of a barrel of the syringe.

26. (canceled)

27. (canceled)

28. The visual aid according to claim 21, wherein the sheet material comprises a plurality of linear perforations arranged such that each adjacent pair of images has a linear perforation separating the pair of images, wherein the linear perforations are arranged to assist a user in tearing a section of the sheet material from a remaining section of the sheet material.

29. The visual aid of claim 21, wherein the visual aid comprises a clip for releasably coupling a first section of the plurality of sections to a second section of the plurality of sections, to obscure a front surface of a third section of the plurality of sections.

30. The visual aid according to claim 21, further comprising a sleeve configured to at least partially receive the sheet material such that the sheet material may slide within the sleeve,
wherein the sleeve has a window formed therein through which at least one image of the plurality of images is visible.

31. The visual aid of claim 30, wherein the plurality of images and the window are arranged such that the plurality of images are sequentially visible through the window as the sheet material is slid through the sleeve.

32. The visual aid according to claim 31, wherein the window and the plurality of images are arranged such that only a single image of the plurality of images may be visible in entirety through the window as the sheet material is slid through the sleeve.

33. The visual aid according to claim 21, comprising a plurality of sheet materials, each sheet material of the plurality of sheet materials having a respective front surface having a corresponding image of the plurality of images arranged thereon,
wherein the plurality of sheet materials are stacked and are releasably coupled together such that each sheet material of the plurality of sheet materials may be uncoupled from the remaining sheet materials.

34. The visual aid according to claim 21, further comprising a tray for retaining the plurality of syringes,
wherein the tray is configured to retain the sheet material such that the plurality of images are visible through the tray.

35. The visual aid of claim 34, wherein the tray comprises a plurality of syringe retainers, each syringe retainer configured to retain a respective syringe of the plurality of syringes, wherein the tray and the sheet material are configured such that each image of the plurality of images is visible through a respective syringe retainer.

36. The visual aid of claim 35, wherein each retainer comprises a recess configured to at least partially receive a respective syringe of the plurality of syringes, wherein at least a portion of each syringe retainer is substantially transparent such that, when the sheet material is retained in the tray, a respective image of the plurality of images is visible through a respective syringe retainer.

37. The visual aid according to claim 21, wherein the visual aid further comprises a plurality of dose interval affordances, each dose interval affordance corresponding to a respective one of the plurality of images and comprising an indication of a time that the volume of medicament indicated in the corresponding image should be administered to a patient.

38. The visual aid according to claim 21, wherein each image indicates a size of the respective syringe.

39. A system comprising:
a plurality of syringes configured to be used in a singular prescribed treatment;
a visual aid for assisting a user in the preparation of the plurality of syringes, the visual aid comprising:
a sheet material comprising a plurality of sections arranged linearly across the sheet material, the sheet material comprising a plurality of images arranged on a front surface of the sheet material, wherein each section of the plurality of sections contains an image of the plurality of images,
wherein each image:
is visually representative of a respective syringe of the plurality of syringes; and
comprises a respective first visual feature that is representative of a respective first visual feature of the respective syringe and a respective second visual feature that is representative of a respective second visual feature of the respective syringe, the respective first visual feature and the respective second visual feature of the image arranged such that they indicate a respective volume of a medicament to be drawn into the respective syringe, wherein each respective volume of medicament represents a given dose in the singular prescribed treatment.

40. A method of using a visual aid for assisting a user in the preparation of a plurality of syringes, the visual aid comprising a sheet material comprising a plurality of sections arranged linearly across the sheet material, the sheet material comprising a plurality of images arranged on a front surface of the sheet material, wherein each section of the plurality of sections contains an image of the plurality of images, wherein each image is visually representative of a respective syringe of the plurality of syringes, and wherein each image comprises a respective first visual feature that is representative of a respective first visual feature of the respective syringe and a respective second visual feature that is representative of a respective second visual feature of the respective syringe, the respective first visual feature and the respective second visual feature of the image arranged such that they indicate a respective volume of a medicament to be drawn into the respective syringe, wherein each respective volume of medicament represents a given dose in a singular prescribed treatment, the method comprising:
using the visual aid to draw a first volume of a medicament into a first syringe of a plurality of syringes.

41. The visual aid of claim 21, wherein each image is visually representative of a given day in the singular prescribed treatment.

42. The visual aid of claim 21, wherein each image of the plurality of images represents a different dose of a same medicament.

43. The visual aid of claim 21, further comprising a third visual feature, wherein the first visual feature comprises a piston that represents a corresponding piston of the respective syringe, the second visual feature comprises a barrel that represents a corresponding barrel of the respective syringe, and the third visual feature comprises an image of a volume of medicament that is to be drawn into the respective syringe.

44. The system of claim 39, wherein the first visual feature comprises a piston that represents a corresponding piston of the respective syringe, the second visual feature comprises a barrel that represents a corresponding barrel of the respective syringe, and a distance between the first visual feature and the second visual feature corresponds to a volume of medicament that is to be drawn into the respective syringe.

45. The system of claim 39, wherein each image indicates a position of a respective piston of the respective syringe relative to a respective barrel of the respective syringe.
